(19) United States
(12) Patent Application Publication
(10) Pub. No.: US 2005/0058670 A1
(43) Pub. Date: Mar. 17, 2005

(54) Oral itraconazole composition which is not affected by ingested food and process for preparing same

(76) Inventors: Jong-Soo Woo, Suwon-si (KR); Hong-Gi Yi, Suwon-si (KR)

Correspondence Address: David A. Einhom, Esq.;Anderson Kill & Olick, P.C., 1251 Avenue of the Americas, New York, NY, 10020, US

(21) Appl. No.: 10/660,859

(22) Filed: Sep. 12, 2003

(51) Int. Cl. A61K 31/4196; A61K 9/00

(52) U.S. Cl. 424/400; 514/383

(57) ABSTRACT

A viscous and glassy composition for oral administration comprising itraconazole, an acidifing agent, an amphiphilic additive, a surfactant and an oil provides a high in vivo bioavailability of itraconazole which is little influenced by ingested food.

### FIELD OF THE INVENTION

The present invention relates to an oral composition of itraconazole having improved itraconazole bioavailability which is not affected by ingested food, and a process for the preparation thereof.

### BACKGROUND OF THE INVENTION

Itraconazole, a triazole compound, is known to have excellent antifungal activity. However, the bioavailability of orally administered itraconazole is very low because it has an exceedingly low solubility of less than 1 μg/ml in water and it remains unionized in the gastric juice due to its pKa value of 3.7. Further, it is known that the degree of bioavailability of orally administered itraconazole varies widely among individuals and depends on other factors such as ingested foods.

PCT International Publication No. WO 85/02767 and U.S. Pat. No. 4,764,604 teach a method for increasing the solubility of itraconazole by employing a cyclodextrin inclusion compound of itraconazole. However, this method has the problems that the incremental increase in the itraconazole solubility is only marginal and various complicated preparative procedures are required.

PCT International Publication No. WO 94/05263 discloses a coated bead preparation, wherein a core made of pharmaceutically inert or neutral sucrose, dextrine, starch and the like is coated with a mixture of itraconazole and a hydrophilic polymer and, then, the resulting bead is coated again with a polymer, e.g., polyethylene glycol. Such a coated bead preparation is commercially available from Janssen Pharmaceutica (Beerse, Belgium) under the trade name of Sporanox capsule. However, the manufacturing process of the above preparation is very complicated due to the fact the beads having an average size of only 600 to 700 μm tend to undergo agglomeration during the manufacturing process. In addition, the itraconazole bioavailability of this preparation varies widely depending on whether it is taken before or after food ingestion.

PCT International Publication No. WO 97/44014 teaches a solid dispersion of itraconazole in a water-soluble polymer, which is prepared by subjecting a mixture of itraconazole and the water-soluble polymer to a melt-extrusion process at a temperature ranging from 245° C. to 265° C. This solid dispersion is described to have an improved bioavailability of itraconazole which is not influenced by ingested foods, and it is commercially available from Janssen Pharmaceutica (Beerse, Belgium) under the trade name of Sporanox tablet. However, the manufacturing process of the solid dispersion is hampered by a number of difficulties in controlling various process variables, and the in vivo bioavailability of itraconazole achievable with the above dispersion is still low.

Sporanox liquid of pH 2, which is prepared by mixing a hydroxypropyl-β-cyclodextrin inclusion compound of itraconazole, hydrochloric acid, propylene glycol, purified water, sodium hydroxide, sodium saccharin and sorbitol, and is commercially available from Janssen Pharmaceutica (Beerse, Belgium), exhibits a high bioavailability of itraconazole when administered before ingestion, but it has the problems that it must be taken in great quantities due to its low itraconazole concentration of 10 mg/ml, the active ingredient rapidly precipitates when it comes in contact with the intestinal juice, and it is effective only against fungus infection of esophagus.

Recently, PCT International Publication No. WO 98/55148 discloses a high viscosity composition comprising a drug which has a very low solubility in water, cyclodextrin, water-soluble acid and a water-soluble organic polymer. However, this composition has a high viscosity, and accordingly, a large amount of a dispersant is used to lower the viscosity during the capsule making process. In addition, the composition exhibits a very low dissolution rate of less than 1% under a neutral or alkaline condition of pH 6.8 or higher.

In this regard, the present inventors suggested a micro-emulsion preconcentrate comprising an antiviral agent which has a very low solubility in water, phosphoric acid, a co-surfactant, a surfactant and an oil in Korean Application No. 2000-83717, and further suggested another micro-emulsion composition modified based on said preconcentrate in Korean Application No. 2001-36930. However, these compositions still exhibit unsatisfactory itraconazole dissolution rates under a neutral or alkaline condition of pH 6.8 or higher, and thus their bioavailabilities of itraconazole more or less depend on ingested food.

### SUMMARY OF THE INVENTION

It is, therefore, a primary object of the present invention to provide an oral composition of itraconazole having improved itraconazole bioavailability which is little influenced by ingested food.

It is a further object of the present invention to provide a process for preparing said oral composition.

In accordance with one aspect of the present invention, there is provided a viscous and glassy composition for oral administration comprising itraconazole, an acidifying agent, an amphiphilic additive, a surfactant and an oil.

In accordance with another aspect of the present invention, there is provided a method of preparing said composition which comprises the steps of: (a) dissolving itraconazole uniformly in a mixture of the acidifying agent, the amphiphilic additive and a volatile solvent, (b) dissolving the surfactant and the oil in the resulting solution, and (c) removing the volatile solvent therefrom.

### BRIEF DESCRIPTION OF THE DRAWINGS

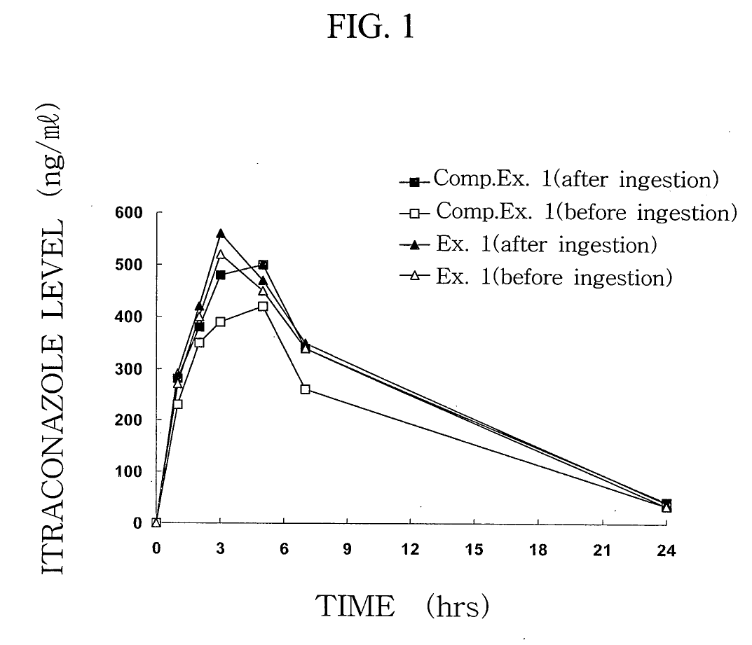

The above and other objects and features of the present invention will become apparent from the following description of the invention, when taken in conjunction with the accompanying drawing, FIG. 1, which shows the respective itraconazole bioavailabilities of the preparations prepared in Example 1 and Comparative Example before and after food ingestion.

### DETAILED DESCRIPTION OF THE INVENTION

The inventive composition comprising itraconazole as an active ingredient may be prepared using the other following components:

(1) Acidifying Agent

Representative examples of the acidifying agent which may be used in the present invention to dissolve itraconazole include phosphoric acid, acetic acid, hydrochloric acid, nitric acid, sulfuric acid, citric acid, fumaric acid, maleic acid, and an aqueous solution thereof, wherein 85% phosphoric acid or a diluted solution thereof is preferred.

(2) Amphiphilic Additive

The amphiphilic additive which is used in the present invention serves to dissolve itraconazole and adjust the viscosity of the composition to a degree suitable for filling into a capsule. Suitable amphiphilic additives that may be used in the present invention include transcutol(diethyleneglycol monoethyl ether, Gattefosse), dimethyl isosorbide(1,4:3,6-dianhydro-2,5-dimethyl-D-glucitol), glycofurol(tetrahydrofurfuryl alcohol polyethylene glycol ether), propylene glycol(1,2-dihydroxypropane), propylene carbonate(4-methyl-2-oxo-1,3-dioxolane), solutol(macrogol 15 hydroxystearate, BASF) and a mixture thereof, wherein transcutol is preferred.

(3) Volatile Solvent

The volatile solvent employed during the manufacturing process, but not present in the final product, assists the dissolution of itraconazole by the action of the acidifying agent. In the present invention, preferably used as the volatile solvent is a non-toxic organic solvent such as an alcohol, e.g., ethanol, propanol and isopropanol, which can be easily volatilized at a temperature of less than 100° C.

(4) Surfactant

The surfactant used in the present invention assists the formulation of a uniform emulsion of an oil and hydrophilic components, and keeps the emulsion stable during storage. Representative examples of the surfactant include:
\mathring{1} polyoxyethylene glycolated natural or hydrogenated vegetable oils such as polyoxyethylene glycolated natural or hydrogenated castor oil (Cremophor, BASF; HCO, Nikkol),
\mathring{2} polyoxyethylene-sorbitan-fatty acid esters wherein fatty acid is mono- or tri-lauric, palmitic, stearic or oleic acid (Tween, ICI), and
\mathring{3} polyoxyethylene fatty acid esters such as polyoxyethylene stearic acid ester (Myrj, ICI).

(5) Oil

The oil component used in the present invention should be compatible with the surfactant and capable of forming a stable microemulsion in an aqueous medium. It may be one of the pharmaceutically acceptable oils such as tocopherols and derivatives thereof including tocopheryl acetate, tocopheryl succinate, and polyethyleneglycol-1000-tocopheryl succinate (TPGS).

The inventive composition is prepared by (a) dissolving itraconazole in a mixture of the acidifying agent, the amphiphilic additive and the volatile solvent, (b) dissolving the surfactant and the oil in the resulting solution, and (c) removing the volatile solvent from the resulting mixed solution.

In step (c), the volatile solvent may be removed by the conventional method, e.g., by heating under the ambient pressure or a vacuum, preferably at a temperature ranging from 40 to 100° C., more preferably at a temperature ranging from 40 to 80° C.

In the above manufacturing process, itraconazole, the acidifying agent, the amphiphilic additive, the volatile solvent, the surfactant and the oil are used in amounts corresponding to a weight ratio in the range of 1:0.5˜15:0.5˜20:0.5˜20:0.5˜15:0.5˜15, preferably 1:1˜10:1˜15:1˜15:1˜10:1˜10.

The final composition of the present invention with the absence of volatile solvent comprises itraconazole, the acidifying agent, the amphiphilic additive, the surfactant and the oil in a weight ratio in the range of 1:0.5˜15:0.5˜20:0.5˜15:0.5˜15, preferably 1:1˜10:1˜15:1˜10:1˜10.

In addition, the inventive composition may comprise pharmaceutically acceptable additives for oral administration such as anti-oxidants.

The pharmaceutical composition of the present invention may be formulated to obtain various pharmaceutical preparations, e.g., a powder, granule, tablet, coated preparation and liquid preparation, in accordance with any of the conventional procedures. For instance, a hard capsule may be prepared by adding a lubricant and other pharmaceutical additives to the pharmaceutical composition, processing the mixture into a powder or granule and filling the powder or granule into a hard gelatin capsule; a tablet, by adding a suitable additive to the pharmaceutical composition and tableting the mixture; a liquid preparation, by dissolving the pharmaceutical composition in water; and a coated preparation, by coating a solution of the pharmaceutical composition on a sugar bead such as Non-pareil (Edward Mendell Co., UK).

The inventive itraconazole composition prepared is transparent and glassy, i.e., it has no fluidity, and has a high viscosity of at least 10,000 cps at 25° C. This is achievable by the combined action of the amphiphilic additive and the volatile solvent used in the inventive method. The high viscosity glassy composition is much more compact as compared with a conventional microemulsion composition.

The inventive composition has self-microemulsifying capability to form high stable and available microemulsion particles when orally administered in the body fluid. Therefore, as the inventive composition can maintain a high and stable level of itraconazole dissolution rate even under a neutral or alkaline condition of pH 6.8 or higher, the itraconazole bioavailability thereof is little influenced by ingested food; the itraconazole bioavailabilities of the inventive composition before and after ingestion are the same, the ratio of AUC_{before ingestion} and AUC_{after ingestion} being close to 1 (AUC: area under the curve of blood concentration), preferably 0.8 or higher.

The following Examples are intended to further illustrate the present invention without limiting its scope.

Further, percentages given below for solid in solid mixture, liquid in liquid, and solid in liquid are on a wt/wt, vol/vol and wt/vol basis, respectively, unless specifically indicated otherwise.

### EXAMPLE 1

### Preparation of Hard Capsule—1)

A hard capsule was prepared using the following ingredients:

| | Quantity(mg/capsule) |
|---|---|
| Itraconazole | 50 |
| Phosphoric acid 85% | 208 |
| Ethanol | 300* |
| Transcutol | 83 |
| Polyoxyethylene-50-hydrogenated | 70 |
| castor oil(HCO 50) | |
| Cremophor EL | 220 |
| dl-α-tocopherol | 60 |

(*not present in the final composition)

Itraconazole was dissolved uniformly in a mixture of 85% phosphoric acid, transcutol and ethanol, and other ingredients were added thereto and dissolved. Then, the resulting mixture was concentrated while heating at 55° C. for 4 hrs to volatilize ethanol therefrom to obtain a viscous and transparent composition having no fluidity. The composition was filled into a hard capsule by the conventional method described in the General Preparation Rule of Korea Pharmacopoeia.

### EXAMPLE 2

### Preparation of Soft Capsule

A soft capsule was prepared using the following ingredients:

| | Quantity(mg/capsule) |
|---|---|
| Itraconazole | 50 |
| Phosphoric acid 85% | 200 |
| Ethanol | 300* |
| Transcutol | 150 |
| Polyoxyethylene-50-hydrogenated | 80 |
| castor oil(HCO 50) | |
| Cremophor EL | 200 |
| dl-α-tocopherol | 60 |

(*not present in the final composition)

The procedure of Example 1 was repeated using the above ingredients to obtain a viscous and transparent composition having no fluidity. The composition was filled into a soft capsule by the conventional method described in the General Preparation Rule of Korea Pharmacopoeia.

### EXAMPLE 3

### Preparation of Hard Capsule—2)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Polyoxyethylene-50-hydrogenated | 200 |
| castor oil(HCO 50) | |
| Cremophor EL | 80 |
| dl-α-tocopherol | 60 |

(*not present in the final composition)

### EXAMPLE 4

### Preparation of Hard Capsule—3)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Polyoxyethylene-50-hydrogenated | 200 |
| castor oil(HCO 50) | |
| Tween 20 | 80 |
| dl-α-tocopherol | 60 |

(*not present in the final composition)

### EXAMPLE 5

### Preparation of Hard Capsule—4)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Polyoxyethylene-50-hydrogenated | 150 |
| castor oil(HCO 50) | |
| Cremophor EL | 150 |
| dl-α-tocopherol | 60 |

(*not present in the final composition)

### EXAMPLE 6

### Preparation of Hard Capsule—5)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Tween 20 | 200 |
| Cremophor EL | 80 |
| dl-α-tocopherol | 60 |

(*not present in the final composition)

### EXAMPLE 7

### Preparation of Hard Capsule—6)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Tween 20 | 150 |
| Cremophor EL | 150 |
| dl-α-tocopherol | 60 |

(*not present in the final composition)

### EXAMPLE 9

### Preparation of Hard Capsule—7)

A hard capsule was prepared by the procedure of Example 1 using the following ingredients:

| | Quantity(mg/capsule) |
|---|---|
| Itraconazole | 50 |
| Phosphoric acid 85% | 200 |
| Ethanol | 300* |
| Transcutol | 83 |
| Polyoxyethylene-50-hydrogenated | 200 |
| castor oil(HCO 50) | |
| Cremophor EL | 80 |
| dl-α-tocopherol | 120 |

(*not present in the final composition)

### EXAMPLE 9

### Preparation of Hard Capsule—8)

A hard capsule was prepared by the procedure of Example 1 using the following ingredients:

| | Quantity(mg/capsule) |
|---|---|
| Itraconazole | 50 |
| Phosphoric acid 85% | 150 |
| Ethanol | 300* |
| Transcutol | 83 |
| Polyoxyethylene-50-hydrogenated | 70 |
| castor oil(HCO 50) | |
| Cremophor EL | 220 |
| dl-α-tocopherol | 60 |

(*not present in the final composition)

### EXAMPLE 10

### Preparation of Hard Capsule—9)

A hard capsule was prepared by the procedure of Example 1 using the following ingredients:

| | Quantity(mg/capsule) |
|---|---|
| Itraconazole | 50 |
| Phosphoric acid 85% | 208 |
| Ethanol | 300* |
| Dimethylisosorbide | 83 |
| Polyoxyethylene-50-hydrogenated | 70 |
| castor oil(HCO 50) | |
| Cremophor EL | 220 |
| dl-α-tocopherol | 60 |

(*not present in the final composition)

### EXAMPLE 11

### Preparation of Hard Capsule—10)

A hard capsule was prepared by the procedure of Example 1 using the following ingredients:

| | Quantity(mg/capsule) |
|---|---|
| Itraconazole | 50 |
| Phosphoric acid 85% | 208 |
| Ethanol | 300* |
| Glycofurol | 83 |
| Polyoxyethylene-50-hydrogenated | 70 |
| castor oil(HCO 50) | |
| Cremophor EL | 220 |
| dl-α-tocopherol | 60 |

(*not present in the final composition)

### EXAMPLE 12

### Preparation of Hard Capsule—11)

A hard capsule was prepared by the procedure of Example 1 using the following ingredients:

| | Quantity(mg/capsule) |
|---|---|
| Itraconazole | 50 |
| Hydrochloric acid | 150 |
| Ethanol | 300* |
| Dimethylisosorbide | 83 |
| Polyoxyethylene-50-hydrogenated | 70 |
| castor oil(HCO 50) | |
| Cremophor EL | 220 |
| dl-α-tocopherol | 60 |

(*not present in the final composition)

### COMPARATIVE EXAMPLE

### Preparation of Hard Capsule

A hard capsule was prepared by the procedure of Example 1, except that the amphiphilic additive, transcutol, was not employed, using the following ingredients:

| | Quantity(mg/capsule) |
|---|---|
| Itraconazole | 50 |
| Phosphoric acid 85% | 208 |
| Ethanol | 300* |
| Polyoxyethylene-50-hydrogenated | 70 |
| castor oil(HCO 50) | |
| Cremophor EL | 220 |
| dl-α-tocopherol | 60 |

(*not present in the final composition)

### TEST EXAMPLE 1

### Dissolution Test

The dissolution rates of itraconazole were determined for the inventive preparation of Example 1; the preparation of Comparative Example; Sporanox capsule; Sporanox tablet; and Sporanox liquid (Janssen Korea), in accordance with the dissolution test method II (paddle method) described in the General Tests chapter of Korean Pharmacopoeia under the conditions listed below:
Test apparatus: Erweka DT80 (Erweka, Germany)
Test solutions: 900 ml of artificial gastric juice (pH 1.2)
900 ml of phosphate buffer (pH 6.8)
Temperature of test solutions: 37±0.5
Rotation speed: 100±4 rpm
Analytical method: liquid chromatography
   column: Cosmosil C18 (150 mm×4.6 mm; Nacalai tesque, Japan)
   mobile phase: acetonitrile/phosphate buffer (pH 7.0)=60:40
   flow rate: 1.2 ml/min.
   detector: UV 255 nm
   injection volume: 10 μl

The amount of dissolved itraconazole is represented by the cumulative amount of itraconazole eluted in 45 min. and the results are shown in Tables 1 and 2.

TABLE 1
Dissolution rates in artificial gastric juice (pH 1.2)

| | | Comparative | Sporanox | Sporanox | Sporanox |
|---|---|---|---|---|---|
| Sample | Example 1 | Example | capsule | tablet | liquid |
| Dissolved | 99.7% | 98.5% | 53.2% | 93.1% | 98.3% |
| amount | | | | | |
| (45 min.) | | | | | |

TABLE 2
Dissolution rates in phosphate buffer(pH 6.8)

| | | Comparative | Sporanox | Sporanox | Sporanox |
|---|---|---|---|---|---|
| Sample | Example 1 | Example | capsule | tablet | liquid |
| Dissolved | 97.2% | 96.3% | 0.5% | 0.5% | 5.7% |
| amount | | | | | |
| (45 min.) | | | | | |

As can be seen in Tables 1 and 2, the preparation of Example 1 exhibits higher amounts of itraconazole dissolved than those of Comparative Example and the commercially available preparations at pH 1.2 or 6.8. In particular, the result shows that, in pH 6.8, the itraconazole dissolution rate of the preparation of Example 1 is remarkably enhanced as compared with the commercially available preparations.

### TEST EXAMPLE 2

### In Vivo Absorption Test

In order to investigate the bioavailability of itraconazole contained in the inventive preparations, in vivo absorption tests were carried out as follows.

Twenty 14 to 15 week-old male Sprague-Dawly rats each weighing about 300 g were fasted for over 48 hours while they were allowed free access to water, and then divided into two groups of 10 each. Subsequently, for the test after food ingestion, ordinary solid feed and water were continuously supplied to the two groups of rats.

The two groups of rats were orally administered with the inventive preparation of Example 1 and the preparation of Comparative Example, respectively, at a dose of 20 mg itraconazole/kg body weight of the rat. Blood samples were taken directly from the hearts of the rats before the administration and after 1, 2, 3, 4, 5, 7 and 24 hours from the administration, and the sera were separated therefrom.

Added to 500 μl each of the serum samples were 50 μl of an internal standard solution (methanol solution containing 500 μg/ml of nitrate econazole) and 200 μl of 1 M carbonate buffer (Ph 10.0). 7 ml of an extraction solvent (n-heptane:isoamylalchol=9:1) was added thereto and the resulting mixture was shaken at 80 rpm for 5 min. to obtain an extract. The extract was centrifuged at 3,000 rpm for 10 min. and the solvent was evaporated at 50° C. under a nitrogen atmosphere. To the resulting residue was added 200 μl of 0.05% triethylamine solution of 65% aqueous acetonitrile and the mixture was subjected to HPLC under the following conditions. The observed results are shown in Table 3 and FIG. 1:
column: Inertsil ODS2 (250×4.6 mm, 5 μm; GL science, Japan)
mobile phase: 65% aqueous acetonitrile solution containing 0.05% triethylamine
detector: UV 258 nm
flow rate: 1.2 ml/min.

injection volume: 100 μl

TABLE 3

| | | | | | AUC_{before ingestion}/ |
|---|---|---|---|---|---|
| | | AUC*¹ | Cₘₐₓ*² | Tₘₐₓ*³ | AUC_{after ingestion} |
| | | (ng · hr/ml) | (ng/ml) | (hr) | (%) |
| Ex. 1 | before | 5697.5 ± 753.6 | 545.6 ± 80.2 | 3.5 ± 1.2 | 94.5% |
| | ingestion | | | | |
| | after | 6023.7 ± 732.5 | 583.5 ± 75.6 | 3.6 ± 1.1 | |
| | ingestion | | | | |
| Comp. Ex. | before | 4532 ± 823.3 | 416.5 ± 73.5 | 4.1 ± 1.5 | 76.1% |
| | ingestion | | | | |
| | after | 5956.6 ± 1023.1 | 535.6 ± 82.6 | 3.8 ± 1.9 | |
| | ingestion | | | | |

*¹Area under the curve of blood concentration till 48 hours
*²Maximum blood concentration
*³Time at the maximum blood concentration

The results in Table 3 and FIG. 1 show that bioavailability of itraconazole observed for the inventive preparation is far less influenced by ingested food as compared to that of Comparative Example.

While the invention has been described with respect to the above specific embodiments, it should be recognized that various modifications and changes may be made to the invention by those skilled in the art which also fall within the scope of the invention as defined by the appended claims.

1. A viscous and glassy composition for oral administration comprising itraconazole, an acidifying agent, an amphiphilic additive, a surfactant and an oil.

2. The composition of claim 1, wherein the itraconazole bioavailability ratio before and after food ingestion is 0.8 or higher.

3. The composition of claim 1, wherein the viscosity measured at 25° C. is at least 10,000 cps.

4. The composition of claim 1 which has self-microemulsifying capability to form microemulsion particles when orally administered in the body fluid.

5. The composition of claim 1, wherein the itraconazole: acidifying agent amphiphilic additive:surfactant:oil ratio by weight is in the range of 1:0.5˜15:0.5˜20:0.5˜15:0.5˜15.

6. The composition of claim 1, wherein the acidifying agent is selected from the group consisting of phosphoric acid, hydrochloric acid and an aqueous solution thereof.

7. The composition of claim 1, wherein the amphiphilic is selected from the group consisting of transcutol, dimethyl isosorbide, glycofurol, propylene glycol, propylene carbonate, solutol and a mixture thereof.

8. The composition of claim 1, wherein the surfactant is selected from the group consisting of polyoxyethylene glycolated natural or hydrogenated vegetable oils, polyoxyethylene-sorbitan-fatty acid esters, polyoxyethylene fatty acid esters and a mixture thereof.

9. The composition of claim 1, wherein the oil is selected from the group consisting of tocopherol, a derivative thereof, and a mixture thereof.

10. A method of preparing the composition of claim 1 which comprises the steps of: (a) dissolving itraconazole uniformly in a mixture of the acidifing agent, the amphiphilic additive and a volatile solvent, (b) further dissolving the surfactant and the oil in the resulting solution, and (c) removing the volatile solvent therefrom.

11. The method of claim 10, wherein the volatile solvent is a C₂ or C₃ alcohol.
